(19) United States
(12) Patent Application Publication
(10) Pub. No.: US 2026/0154815 A1
(43) Pub. Date: Jun. 4, 2026

(54) RISK PREDICTION OF HEART FAILURE-RELATED MORTALITY

(71) Applicants: Case Western Reserve University, Cleveland, OH (US); University Hospitals Cleveland Medical Center, Cleveland, OH (US)

(72) Inventors: Shuo Li, Cleveland, OH (US); Sadeer Al-Kindi, Manvel, TX (US); Sanjay Rajagopalan, Shaker Heights, OH (US)

(21) Appl. No.: 19/229,421

(22) Filed: Jun. 5, 2025

Related U.S. Application Data
(60) Provisional application No. 63/726,769, filed on Dec. 2, 2024.

(51) Int. Cl. G06T 7/00; A61B 5/00; G06T 7/11; G06V 10/25; G06V 10/26; G06V 10/40; G06V 10/774; G16H 10/60

(52) U.S. Cl. CPC G06T 7/0012; A61B 5/0044; G06T 7/11; G06V 10/25; G06V 10/26; G06V 10/40; G06V 10/774; G16H 10/60; G06T 2207/10088; G06T 2207/20081; G06T 2207/30048; G06V 2201/031

(57) ABSTRACT

The present disclosure, in some embodiments, relates to a method. The method includes accessing image data stored in a memory. The image data corresponds to a patient. A plurality of pathophysiological mechanism related features are extracted from the image data. The plurality of pathophysiological mechanism related features correspond to one or more pathophysiological mechanisms relating to heart failure. The plurality of pathophysiological mechanism related features are provided to a machine learning component. The machine learning component is configured to generate a medical prognosis for the patient using the plurality of pathophysiological mechanism related features.

### CROSS REFERENCE TO RELATED APPLICATIONS

This application claims the benefit of U.S. Provisional Application 63/726,769 filed Dec. 2, 2024 and entitled IMAGING RADIOMICS FOR PREDICTING RISK OF HEART FAILURE, the contents of which is hereby incorporated by reference in its entirety.

### BACKGROUND

In recent years, the development of machine vision tools for analyzing medical images has gained substantial attention. Machine vision tools include computer systems utilizing artificial intelligence to interpret medical imaging data, offering significant potential to transform patient care and improve healthcare.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of a medical assessment apparatus comprising a machine learning component configured to utilize pathophysiological mechanism related features to generate a medical prognosis of cardiac dysfunction.

FIG. 2 illustrates some other embodiments of a medical assessment apparatus comprising a machine learning component configured to utilize pathophysiological mechanism related features to generate a medical prognosis of cardiac dysfunction.

FIG. 3 illustrates a table showing some exemplary pathophysiological mechanism related features that may be utilized by a disclosed medical assessment apparatus to generate a medical prognosis of cardiac dysfunction.

FIG. 4 illustrates a flow diagram showing some embodiments of a method of utilizing pathophysiological mechanism related features to generate a medical prognosis of cardiac dysfunction for a patient.

FIG. 5 illustrates various exemplary images relating to the extraction of pathophysiological mechanism related features corresponding to fibrosis within a heart.

FIG. 6 illustrates various exemplary images corresponding to a disclosed automated extraction of pathophysiological mechanism related features.

FIG. 7 illustrates some additional embodiments of a medical assessment apparatus comprising a machine learning component configured to utilize a plurality of pathophysiological mechanism related features and clinical information to generate a medical prognosis of cardiac dysfunction for a patient.

FIG. 8 illustrates some additional embodiments of a medical assessment apparatus comprising a machine learning component configured to utilize a plurality of pathophysiological mechanism related features, clinical information, and/or demographic information to generate a medical prognosis of cardiac dysfunction for a patient.

FIG. 9 illustrates a flow diagram showing some additional embodiments of a method of utilizing pathophysiological mechanism related features, clinical information, and/or demographic information to generate a medical prognosis of cardiac dysfunction for a patient.

FIG. 10 illustrates a block diagram of some embodiments of a system comprising a machine learning component configured to utilize a plurality of pathophysiological mechanism related features to generate a medical prognosis of cardiac dysfunction for a patient.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Heart failure occurs when the heart muscle doesn't pump blood as well as it should, causing the heart to fail to meet the body's needs. Heart failure can be caused by certain heart conditions that gradually leave a heart too weak and/or stiff to fill and pump blood properly. These conditions include narrowed arteries, high blood pressure, obesity, diabetes, and/or the like. Heart failure affects a large number of people in the United States and is associated with significant mortality and morbidity, similar to some types of cancers.

Patients that suffer from heart failure may be high risk (e.g., more advanced heart failure) or low risk (e.g., less advanced heart failure). Intensive medical treatments (e.g., surgical heart pumps, transplants, etc.) may be implemented for high-risk patients and less intensive medical treatments may be implemented for low-risk patients to improve systems of heart failure. There is a compelling need for a personalized heart failure risk prediction that includes a time to heart failure-related mortality prediction for patients. If a patient's time to heart failure-related mortality is known (e.g., a time the patient may experience heart failure-related mortality from a first diagnosis of heart failure), then the patient may receive precise medical treatment strategies, tailored to their degree of heart failure, to improve the symptoms of heart failure and extend or eliminate the time to heart failure-related mortality. However, currently there are no widely validated models for heart failure risk that predict mortality in patients with heart failure. For instance, under current variable-based heart failure risk prediction models, high-risk patients may be referred to intensive medical treatments too late and low-risk patients that are referred to intensive medical treatments may be adversely affected. Thus, the lack of validated models for heart failure mortality risk prediction hinders timely initiation of precise treatments for patients having heart failure.

Accordingly, the present disclosure relates to an apparatus and/or method that utilizes a plurality of pathophysiological mechanism related features extracted from digital images (e.g., cardiovascular magnetic resonance (CMR) images) to generate a medical prognosis relating to heart failure mortality risk. In some embodiments, the method may access image data (e.g., comprising the digital images) stored in a memory. A plurality of pathophysiological mechanism related features are extracted from the image data. The plurality of pathophysiological mechanism related features correspond to one or more pathophysiological mechanisms relating to heart failure-related morality. The plurality of pathophysiological mechanism related features are provided to a machine learning component, which is configured to use the features to generate a medical prognosis relating to heart failure mortality risk. By utilizing the plurality of pathophysiological mechanism related features to generate a medical prognosis relating to heart failure mortality risk, the disclosed apparatus and/or method may provide health care professionals with a tool that can identify a degree of heart failure in patients to provide timely and precise treatments for the patients.

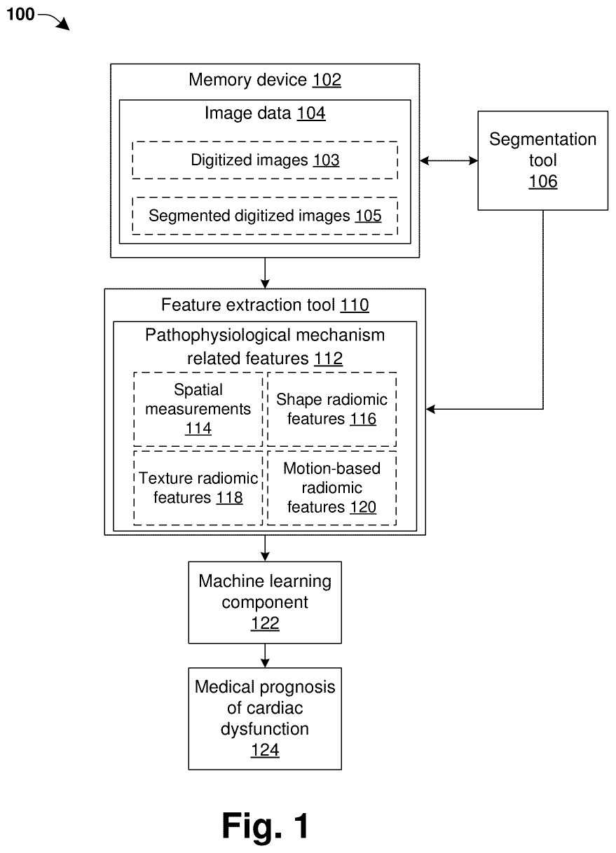

FIG. 1 illustrates some embodiments of a medical assessment apparatus 100 comprising a machine learning component configured to utilize pathophysiological mechanism related features to generate a medical prognosis of cardiac dysfunction.

The medical assessment apparatus 100 comprises a memory device 102 configured to store image data 104 of a patient that may have cardiac dysfunction (e.g., heart failure). The image data 104 comprises one or more digitized images 103. In some embodiments, the one or more digitized images 103 may comprise one or more magnetic resonance (MR) images, one or more cardiovascular magnetic resonance (CMR) images), and/or the like. In further embodiments, the one or more digitized images 103 comprises one or more computed tomography (CT) images, one or more cardiac CT images, one or more contrast enhanced CT images, and/or the like.

In some embodiments, a segmentation tool 106 is in communication with the memory device 102. The segmentation tool 106 is configured to segment the image data 104 (e.g., the one or more digitized images 103) to generate one or more segmented digitized images 105 that respectively identify one or more regions of interest (e.g., volumes of interest). In some embodiments, the one or more regions of interest may comprise one or more views and/or regions of a heart of the patient. For example, the one or more regions of interest may include views of muscle tissue of a heart, a two chamber view/region of a heart, a three chamber view/region of a heart, a four chamber view/region of a heart, a short axis view of a heart, a long axis view of a heart, and/or the like. In some embodiments, the one or more segmented digitized images 105 may be stored in the memory device 102 as part of the image data 104.

A feature extraction tool 110 is configured to extract a plurality of pathophysiological mechanism related features 112 from the image data 104 (e.g., from the one or more segmented digitized images 105). The plurality of pathophysiological mechanism related features 112 are features that characterize mechanisms linked with heart failure, and outcomes such as, for example, heart failure-related mortality. For example, the plurality of pathophysiological mechanism related features 112 may include features that characterize the different regions of interest and/or changes (e.g., functional changes) in the different regions of interest that may be indicative of heart failure and/or of heart failure-related mortality. In some embodiments, the plurality of pathophysiological mechanism related features 112 may include spatial measurements 114, shape radiomic features 116, texture radiomic features 118, and motion-based radiomic features 120 extracted from the one or more regions of interest. In various embodiments, the plurality of pathophysiological mechanism related features 112 may include first-order radiomic features (e.g., radiomic features generated on a single imaging unit values within a region of interest) and/or second-order radiomic features (e.g., radiomic features generated based on associations between neighboring imaging unit values within a region of interest) that have been selected to correspond to one or more pathophysiological mechanisms indicative of heart failure and/or heart failure-related mortality risks.

The plurality of pathophysiological mechanism related features 112 include features that correspond to pathophysiological mechanisms for heart failure. In some embodiments, the plurality of pathophysiological mechanism related features 112 include one or more of cardiac size/remodeling features (e.g., features relating to LV volumes/mass, LA volume, RV size, RA size, etc.), cardiac function features (e.g., features relating to LV strain, LA function/strain, RA function/strain, etc.), cardiac fibrosis features (e.g., features relating to a presence, size, and characteristics of scar tissue).

A machine learning component 122 is configured to utilize the plurality of pathophysiological mechanism related features 112 to generate a medical prognosis of cardiac dysfunction 124. In some embodiments, the medical prognosis of cardiac dysfunction 124 is related to heart failure risk (e.g., heart failure-related mortality risk) for the patient. In various embodiments, the machine learning component 122 comprises one or more machine learning models trained to generate the medical prognosis of cardiac dysfunction 124 based on the plurality of pathophysiological mechanism related features 112.

In some embodiments, the generated medical prognosis of cardiac dysfunction 124 may comprise a prediction or determination of a degree of heart failure the patient suffers from and/or a time-to-event metric for the patient. In various embodiments, the degree of heart failure the patient suffers from may include high-risk heart failure or low risk heart failure. In various embodiments, the time-to-event metric for the patient may include a time to heart failure-related mortality, a time to more advanced heart failure, a time to a high-risk cardiac event (e.g., a time to a heart attack), etc. For example, the time to heart failure related morality is defined as a time interval from a first time (e.g., a time in which a patient first receives a heart failure diagnosis, a time from a scan identifying heart failure, etc.) to a mortality event (e.g., death of the patient) attributable to cardiac dysfunction associated with heart failure. The time to heart failure related mortality provides healthcare professionals the ability to provide the patient with precise and timely medical treatments, tailored to their degree of heart failure, to improve the symptoms of heart failure and to extend or eliminate the time to heart failure-related mortality. It has been appreciated that utilizing features that are related to different pathophysiological mechanisms, the medical prognosis of cardiac dysfunction 124 may have improved accuracy and time-to-event estimation capability compared to existing models. Therefore, the disclosed medical assessment apparatus 100 may provide healthcare professionals the ability to identify a severity of heart failure in patients and that will benefit from timely personalize medical treatment based on the severity of the heart failure.

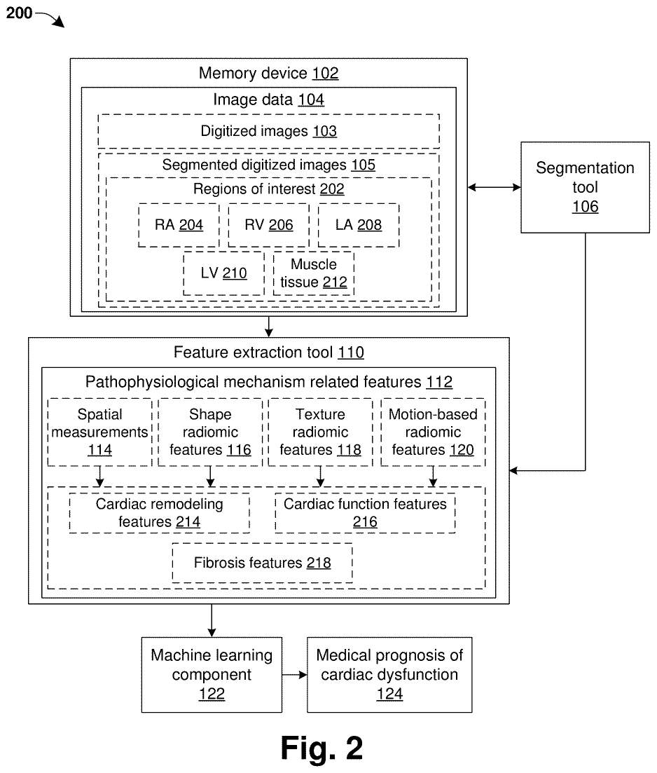

FIG. 2 illustrates some other embodiments of a medical assessment apparatus 200 comprising a machine learning component configured to utilize pathophysiological mechanism related features to generate a medical prognosis of cardiac dysfunction.

The medical assessment apparatus 200 comprises a memory device 102 configured to store image data 104 for a patient that may be susceptible to cardiac dysfunction (e.g., heart failure). In some embodiments, the image data 104 may comprise one or more digitized images 103. The one or more digitized images 103 may comprise one or more cardiovascular magnetic resonance (CMR) images. For example, the one or more digitized images 103 may comprise cine magnetic resonance (MR) images, Late Gadolinium Enhancement (LGE) MR images, and/or the like. In some embodiments, the one or more digitized images 103 may comprise non-contrast MR images (e.g., cine MR images) and contrast MR images (LGE MR images). In further embodiments, the one or more digitized images 103 may comprises non-contrast images (e.g., cine MR images) and does not comprise contrast images. In some embodiments, the memory device 102 may comprise electronic memory, such as solid state memory, static random-access memory (SRAM), dynamic random-access memory (DRAM), and/or the like.

In some embodiments, a segmentation tool 106 is in communication with the memory device 102. The segmentation tool 106 is configured to segment the one or more digitized images 103 to generate one or more segmented digitized images 105 that respectively identify one or more regions of interest 202 (e.g., volumes of interest (VOI)). The one or more regions of interest 202 may, for example, comprise heart tissue (e.g., a heart), one or more regions (e.g., chambers) of a heart, muscle tissue (e.g., myocardial tissue), one or more other tissues (e.g., liver tissue, stomach tissue, etc.), and/or the like. In some embodiments, the one or more regions of interest 202 may comprise chambers of the heart, such as right atrium (RA) 204, right ventricle (RV) 206, left atrium (LA) 208, and left ventricle (LV) 210. The one or more regions of interest 202 may, for example, further comprise muscle tissue 212, heart valves (e.g., pulmonary valve, aortic valve, tricuspid valve, etc.), artery tissue (e.g., right coronary artery, aorta, branches of the aorta, pulmonary artery, branches of the pulmonary artery, etc.), and/or the like.

In some embodiments, the one or more segmented digitized images 105 may comprise and/or be one or more binary masks. In some such embodiments, the one or more binary masks comprise images having a value of “1” in image units (e.g., pixels, voxels, etc.) identified as being within the one or more regions of interest 202 and having a value of “0” in image units outside of the one or more regions of interest 202.

In some embodiments, the segmentation tool 106 may comprise a deep learning model. For example, the segmentation tool 106 may comprise a graphical neural network (GNN). In some embodiments, the segmentation tool 106 may utilize transformer models, which use a self-attention mechanism to differentially weigh a significance of each input data element. The self-attention mechanism may improve a GNN's ability to formulate geometric and semantic relationships between the regions of interest 202 (e.g., VOIs). In some embodiments, the deep learning model may be run on one or more processors (e.g., a central processing unit including one or more transistor devices configured to operate computer code to achieve a result, a microcontroller, or the like).

In some embodiments, the segmentation tool 106 may comprise a first segmentation module and a second segmentation module. The first segmentation module may, for example, be configured to segment one or more first digitized images (e.g., cine MR images) of the digitized images 103 and generate one or more first segmented digitized images that respectively identify one or more first regions of interest (e.g., four chambers of a heart). The first segmentation module may be configured to perform spatial and temporal segmentation on the one or more first digitized images to generate the one or more first segmented digitized images. In some embodiments, the one or more first segmented digitized images are part of the segmented digitized images 105. The one or more first digitized images may, for example, include two chamber cine MR images of a heart, three chamber cine MR images of a heart, fourth chamber cine MR images of a heart, and/or the like. The second segmentation module may, for example, be configured to segment one or more second digitized images (e.g., Late Gadolinium Enhancement (LGE) MR images) of the digitized images 103 and generate one or more second segmented digitized images that respectively identify one or more second regions of interest (e.g., muscle tissue, one or more chambers of a heart, etc.). The one or more second digitized images may, for example, include one or more LGE MR images. In some embodiments, the one or more second segmented digitized images are part of the segmented digitized images 105. In yet further embodiments, the second segmentation module is configured to segment and/or identify locations of scar tissue in a heart. The first segmentation module and the second segmentation module may, for example, each comprise a deep learning model and/or may each utilized transformer models.

A feature extraction tool 110 is configured to extract a plurality of pathophysiological mechanism related features 112 from the image data 104 (e.g., from the one or more segmented digitized images 105). The plurality of pathophysiological mechanism related features 112 may include features that have been selected to correspond to pathophysiological mechanisms indicative of heart failure and/or associated with heart failure-related mortality risk. The plurality of pathophysiological mechanism related features 112 may include spatial measurements 114, shape radiomic features 116, texture radiomic features 118, and/or motion-based radiomic features 120 taken from the one or more regions of interest 202. In some embodiments, the feature extraction tool 110 may be implemented as a computer code run by a processing unit (e.g., a central processing unit including one or more transistor devices configured to operate computer code to achieve a result, a microcontroller, or the like).

The spatial measurements 114 may include morphological features (e.g., volume, area, etc.), mass features (e.g., mass, center of mass, etc.), depot-specific assessments (e.g., slice cross-sectional areas, thicknesses, etc.), and/or the like. The spatial measurements 114 may further include statistical measurements (e.g., mean, median, standard deviation, maximum, minimum, etc.) taken over a plurality of regions of interest and/or segmented digitized images.

In some embodiments, the shape radiomic features 116 may include features related to and/or derived from elongation, flatness, least axis length, major axis length, maximum 2D diameter column, maximum 2D diameter row, maximum 2D diameter slice, maximum 3D diameter, mesh volume, minor axis length, sphericity, surface area, surface volume ratio, voxel volume, and/or the like. The texture radiomic features 118 may include features related to and/or derived from first-order features, Gray Level co-occurrence Matrix, Gray Level Size zone Matrix, Gray Level Run Length Matrix, Neighboring Gray Tone Difference Matrix, Gray Level Dependence Matrix, and/or the like. The motion-based radiomic features 120 may include cardiac motion features (e.g., strain, deformation, and/or acceleration of one or more regions of the heart), motion statistics (e.g., peak strain, mean displacement, standard deviation of strain, etc.), abnormal cardiac motion features (e.g., abnormal motion of muscle tissue in a heart corresponding to scarring). In some embodiments, the cardiac motion features may include 2D motion of a heart and/or 3D motion of a heart. Further, the motion-based radiomic features 120 may be extracted by, for example, tracking motion of one or more regions of interest in a plurality of segmented digitized images and computing motion-based features from the plurality of segmented digitized images. In some embodiments, the feature extraction tool 110 comprises a motion extraction module configured to extract the motion-based radiomic features 120. In various embodiments, the motion extraction module may be configured to determine a presence, size, and/or location of scar tissue from a segmented digitized image that identifies a region of interest (e.g., the myocardium) suspected of having the scar tissue.

In some embodiments, the plurality of pathophysiological mechanism related features 112 (e.g., the spatial measurements 114, the shape radiomic features 116, the texture radiomic features 118, and the motion-based radiomic features 120) may include one or more of cardiac remodeling features 214 (e.g., features relating to changes in a shape, size, volume, mass, and/or geometry of a heart), cardiac function features 216 (e.g., features relating to functions, contractions, and/or strain of a heart), fibrosis features 218 (e.g., features relating to a presence, size, and characteristics of scar tissue). In some embodiments, each feature may be associated with a specific aspect of heart failure and/or of heart failure-related mortality risk. For example, the cardiac remodeling features 214 may be associated with structural deterioration of the heart (e.g., due to chronic hypertension); the cardiac function features 216 may be associated with associated with weakened contractility (e.g., reduce blood pumped by the heart per each beat) and/or arrhythmia susceptibility; and the fibrosis features 218 may be associated with myocardial scarring. In various embodiments, the texture radiomic features 118 and/or the motion-based radiomic features 120 may be associated with myocardial scarring, LV strain, exercise peak oxygen consumption and heart filling pressures, each of which are prognostic markers for heart failure-related mortality risk.

In some embodiments, the plurality of pathophysiological mechanism related features 112 (e.g., the spatial measurements 114, the shape radiomic features 116, the texture radiomic features 118, and the motion-based radiomic features 120) may further include one or more of contractility features (e.g., features relating to contractions of a heart, ejection fraction, wall thickening, etc.) and tissue heterogeneity features (e.g., features relating to a uniformity or variation of the composition and/or structure of heart tissue). In further embodiments, the plurality of pathophysiological mechanism related features 112 may include features related to other pathophysiological mechanisms (e.g., inflammation, arrhythmias, etc.).

A machine learning component 122 is configured to utilize the plurality of pathophysiological mechanism related features 112 to generate a medical prognosis of cardiac dysfunction 124. In some embodiments, the medical prognosis of cardiac dysfunction 124 may provide for an indication of a degree of heart failure and/or a time to heart failure (e.g., a time interval from a MRI scan to a mortality event). In some embodiments, the machine learning component 122 may comprise a regression model, a Cox Hazard regression model, a support vector machine (SVM), a linear discriminant analysis (LDA) classifier, a Random Forest, or the like. In some embodiments, the machine learning component 122 may be run on one or more processors (e.g., a central processing unit including one or more transistor devices configured to operate computer code to achieve a result, a microcontroller, or the like.

By using the plurality of pathophysiological mechanism related features 112 to generate the medical prognosis of cardiac dysfunction 124, a heart failure mortality risk can be accurately assessed leading to improved treatment for patients. In various embodiments, the medical assessment apparatus 100 further includes a patient treatment component configured to recommend and/or apply a medical treatment to the patient based on the generated medical prognosis of cardiac dysfunction 124. Further, the medical prognosis of cardiac dysfunction 124 includes information related to whether a patient suffers from high-risk heart failure or low-risk heart failure and a time to heart failure-related mortality, thereby facilitating the patient getting accurate and timely personalized medical treatments. For example, early identification of high-risk heart failure in a patient may allow for the consideration and/or application of advanced treatments, such as pharmacotherapies (e.g., soluble guanylate cyclase stimulators), devices (e.g., resynchronization pacemakers, defibrillators, contractility modulation devices), and interventional options (e.g., mechanical circulatory support, heart transplantation). It has been appreciated that patients with high-risk heart failure may be diagnosed late and/or are referred late (e.g., due to inaccuracies in existing medical assessment models) for the aforementioned treatments, thereby leading to increased morality and poor treatment outcomes. Furthermore, accurate detection of low-risk heart failure in a patient mitigates the application of intensive and costly treatments that should be applied to patients with high-risk heart failure. This mitigates potential averse outcomes for the patient with low-risk heart failure and mitigates misallocation of resources.

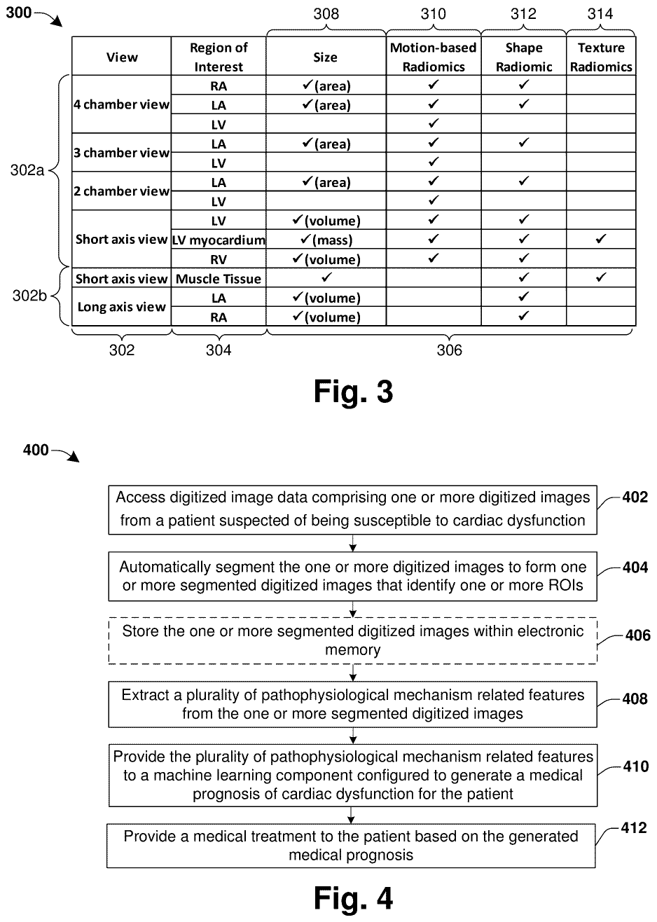

FIG. 3 illustrates a table 300 showing some exemplary pathophysiological mechanism related features that may be utilized by a disclosed medical assessment apparatus to generate a medical prognosis of cardiac dysfunction. It will be appreciated that the pathophysiological mechanism related features shown in table 300 are merely examples of pathophysiological mechanism related features that may be used by a disclosed medical assessment apparatus and are not limiting.

Table 300 includes features that convey specific facets of heart failure mortality risk that consolidate imaging analogs of pathophysiological mechanisms relevant to heart failure. Table 300 illustrates different regions of interest 304 taken at different views 302 and associated with pathophysiological mechanism related features 306. The different views 302 may include one or more digitized images (e.g., MR images) of a specific view and/or orientation of a heart. In some embodiments, the different views 302 comprises a first subset of views 302a including one or more first digitized images (e.g., cine MR images) and a second subset of views 304b including one or more second digitized images (e.g., LGE MR images). In various embodiments, the one or more first digitized images are non-contrast MR images and the one or more second digitized images are contrast MR images. The different regions of interest 304 may include regions of interest that have been identified in a segmented digitized image (e.g., a segmented MR image) in the corresponding view 302. The pathophysiological mechanism related features 306 associated with each region of interest include one or more of size features 308, motion-based radiomic features 310, shape radiomic features 312, and texture radiomic features 314. For example, pathophysiological mechanism related features associated with a right atrium (RA) of a heart, where the corresponding digitized image of the RA is taken from a four chamber view of the heart, may include an area of the RA, motion-based radiomic features extracted from the RA, and shape radiomic features extracted from the RA.

FIG. 4 illustrates a flow diagram showing some embodiments of a method 400 of utilizing pathophysiological mechanism related features to generate a medical prognosis of cardiac dysfunction for a patient.

While the disclosed methods (e.g., methods 400 and/or 900) are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 402, digitized image data comprising one or more digitized images from a patient suspected of being susceptible to cardiac dysfunction (e.g., heart failure) is accessed. In some embodiments, the digitized image data may comprise one or more magnetic resonance (MR) images. In some embodiments, the patient may have high blood pressure, high cholesterol, obesity, diabetes, a high body mass index (BMI), and/or the like.

At act 404, the one or more digitized images are automatically segmented to form one or more segmented digitized images that identify one or more regions of interest (ROIs). In some embodiments, the one or more RIOs may comprise one or more of heart tissue, muscle tissue, one or more chambers of a heart, such as a right atrium (RA), a right ventricle (RV), a left atrium (LA), a left ventricle (LV) of a heart, and/or the like. In some embodiments, the one or more digitized images may be segmented using a deep learning model.

At act 406, the one or more segmented digitized images may be stored within electronic memory, in some embodiments.

At act 408, a plurality of pathophysiological mechanism related features are extracted from the one or more segmented digitized images. In some embodiments, the plurality of pathophysiological mechanism related features comprise one or more of spatial measurements, shape radiomic features, texture radiomic features, and motion-based radiomic features extracted from the ROIs.

At act 410, the plurality of pathophysiological mechanism related features are provided to a machine learning component configured to generate a medical prognosis of cardiac dysfunction (e.g., heart failure mortality risk) for the patient.

At act 412, a medical treatment is provided to the patient based on the generated medical prognosis.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, process) cause the machine to performed the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

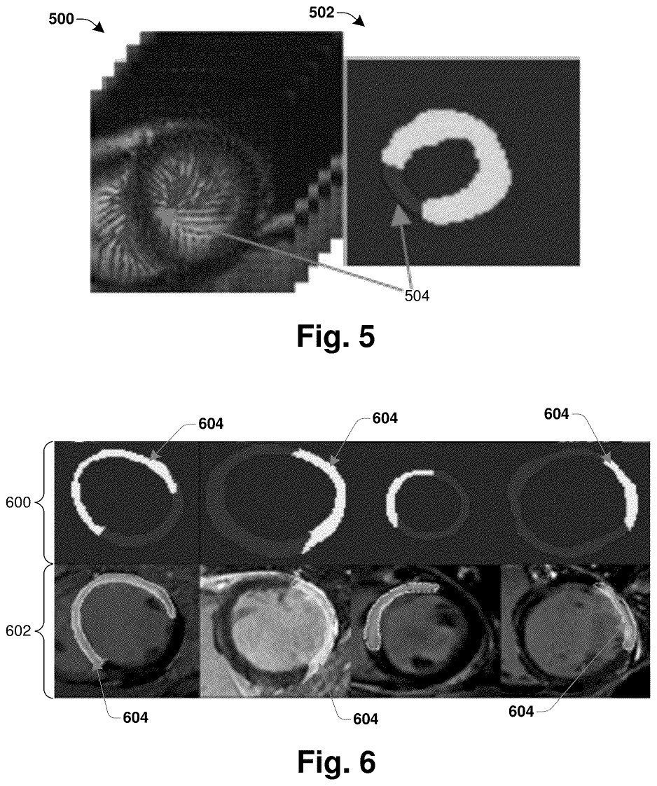

FIG. 5 illustrates various exemplary images 500-502 relating to the extraction of pathophysiological mechanism related features corresponding to fibrosis within a heart.

Image 500 shows a plurality of digitized images of a heart comprising scar tissue 504. In some embodiments, the plurality of digitized images may comprise cine MR image data (e.g., a plurality of digitized images, each image corresponding to a distinct point in in a cardiac cycle of a heart). Image 502 shows an annotated digitized image identifying a location and/or size of the scar tissue 504. The disclosed method and apparatus are configured to extract features relating to the scar tissue 504. In various embodiments, the scar tissue 504 may be extracted from muscle tissue (e.g., the myocardium) of a heart. In various embodiments, some extracted features relating to the fibrosis within the heart include a presence, a location, and/or a size of the scar tissue 504.

In various embodiments, the plurality of digitized images in the image 500 are provided to a disclosed feature extraction tool (e.g., feature extraction tool 110 of FIG. 1). The plurality of digitized images may, for example, be segmented digitized images that identify one or more ROIs in a heart (e.g., muscle tissue of the heart). The disclosed feature extraction tool is configured to calculate a motion or flow of voxels for the one or more ROIs between consecutive images in the plurality of digitized images. Velocity maps may be calculated to represent the displacement of voxels between consecutive images in the plurality of digitized images. In some embodiments, the velocity maps may provide for a speed and direction of motion of the one or more ROIs across the plurality of digitized images, where the motion may indicate areas of abnormalities (such as scarring) in the one or more ROIs. As a result, motion-based radiomic features may be extracted by the disclosed feature extraction tool from the plurality of digitized images in the image 500. In further embodiments, the disclosed feature extraction tool may be configured to determine an absence or presence of scar tissue in a heart. It will be appreciated that the disclosed feature extraction tool may further extract cardiac function features from a heart by calculating the motion or flow of voxels for one or more other ROIs between consecutive images in another plurality of digitized images.

FIG. 6 illustrates various exemplary images 600-602 illustrating a correlation between the disclosed automated extraction of pathophysiological mechanism related features corresponding to fibrosis within a heart and manual segmentation and/or extraction of features corresponding to fibrosis.

A plurality of first images 600 illustrate annotated digitized images identifying a location and/or area of scar tissue 604 in different regions of a heart. The scar tissue 604 is represented in green, whereas non-scar tissue of the heart is represented in red in the plurality of first images 600. In some embodiments, a location and/or area of the scar tissue 604 in the plurality of first images 600 is/are extracted by a disclosed feature extraction tool. A plurality of second images 602 illustrate annotated digitized images that comprise manually segmented scar tissue in different regions of a heart (e.g., from ground truth LGE images). In some embodiments, the location and/or area of the scar tissue 604 in the plurality of first images 600 is/are extracted from cine MR image data. In the plurality of second images 602, the yellow line represents a manually segmented area of the scar tissue 604 and the green area in each of the plurality of second images 602 provides an overlay of the location and area of the scar tissue 604 that was automatically extracted by the disclosed feature extraction tool. In various embodiments, automatically extracted features (e.g., scar tissue location, presence, area, size, etc.) relating to the fibrosis within the heart by the disclosed feature extraction tool from non-contrast MR images (e.g., from cine MR images) is able to achieve a very close correlation with manual segmentation and extraction of features relating fibrosis (e.g., having an accuracy above 90%).

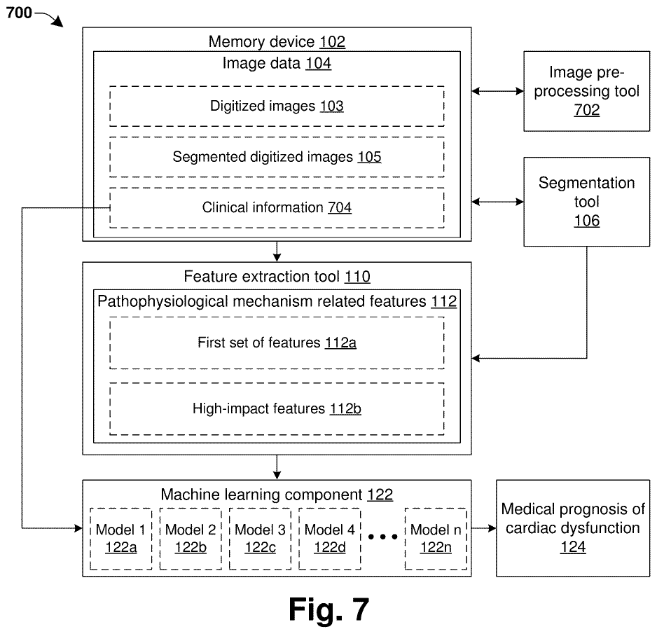

FIG. 7 illustrates some additional embodiments of a medical assessment apparatus 700 comprising a machine learning component configured to utilize a plurality of pathophysiological mechanism related features and clinical information to generate a medical prognosis of cardiac dysfunction for a patient.

The medical assessment apparatus 700 comprises a memory device 102 configured to store image data 104. The image data 104 includes one or more digitized images 103 for a patient that may be susceptible to cardiac dysfunction (e.g., heart failure). The one or more digitized images 103 may, for example, comprise one or more non-contrast digitized images (e.g., non-contrast MR images), one or more contrast digitized images (e.g., contrast MR images), and/or the like. A segmentation tool 106 is configured to operated upon the one or more digitized images 103 to form one or more segmented digitized images 105. In some embodiments, the one or more segmented digitized images 105 may be stored in the memory device 102 as part of the one or more digitized images 103.

In some embodiments, an image pre-processing tool 702 is in communication with the memory device 102. The image pre-processing tool 702 is configured to perform one or more pre-processing steps on the one or more digitized images 103 to improve image quality and/or subsequent feature extraction. In some embodiments, the image pre-processing tool 702 is configured to apply data harmonization to the one or more digitized images 103 to reduce scanner variability (e.g., from images acquitted from different scanners or imaging protocols) that can otherwise interfere with accurate and precise feature extraction. In some embodiments, the image pre-processing tool 702 is further configured to perform resampling on the one or more digitized images 103, such that the one or more digitized images 103 are resampled to a predefined size. The image pre-processing tool 702 may, for example, achieve the resampling by utilizing Lanczos interpolation and/or the like. In some embodiments, the image pre-processing tool 702 is further configured to apply filtering to mitigate deviations in flat image regions.

A feature extraction tool 110 is configured to extract a plurality of pathophysiological mechanism related features 112 from the one or more segmented digitized images 105. The plurality of pathophysiological mechanism related features 112 may include features that have been selected to correspond to pathophysiologic mechanisms indicative of heart failure and/or heart failure-related mortality risks. The plurality of pathophysiological mechanism related features 112 may include one or more of spatial measurements, shape radiomic features, texture radiomic features, and motion-based radiomic features taken from different regions of interest within the one or more segmented digitized images 105.

In some embodiments, during training the feature extraction tool 110 is configured to extract a first set of features 112a that include a first plurality of features. From the first set of features 112a, the feature extraction tool 110 may be configured to identify a plurality of high-impact features 112b that include a second plurality of features that is less than the first plurality of features. In some embodiments, the feature extraction tool 110 may identify the plurality of high-impact features 112b within the context of a time-to-event model (e.g., a Cox proportional hazard model). In some embodiments, the high-impact features 112b may include scar tissue texture, cardiac motion, LA volumes, global longitudinal strain (GLS), and/or the like. In various embodiments, after training is completed the feature extraction tool 110 may extract the plurality of pathophysiological mechanism related features 112 to be or to predominantly be the high-impact features 112b.

A machine learning component 122 is configured to utilize the plurality of pathophysiological mechanism related features 112 (e.g., the high-impact features 112b) to generate a medical prognosis of cardiac dysfunction 124.

In some embodiments, the memory device 102 is further configured to store clinical information 704 relating to the patient. In various embodiments, the clinical information 704 may include one or more of systolic blood pressure, diastolic blood pressure, diabetes, chronic kidney disease, smoking status, cardiovascular risk factors, heart failure subtype, body mass index (BMI), B-type Natriuretic Peptide (BNP), and/or the like. In some embodiments, the heart failure subtype may include HFrEF (Heart Failure with reduced Ejection Fraction), HFpEF (Heart Failure with preserved Ejection Fraction), HFmrEF (Heart Failure with mildly reduced Ejection fraction), HFimpEF (Heart Failure with improved Ejection Fraction), or the like.

It has been appreciated that a patient's clinical information may affect the pathophysiological mechanisms that related to heart failure and/or heart failure-related mortality risks for that patient. For example, a patient that is a smoker may have different pathophysiological mechanisms relating to heart failure than a non-smoker. In another example, a patient with a first heart failure subtype (e.g., HFrEF) may have some different pathophysiological mechanisms relating to heart failure than another patient with a second heart failure subtype (e.g., HFpEF). The clinical information 704 stored in the memory device 102 may therefore also affect the pathophysiological mechanism related features 112 used by the machine learning component 122 and/or a weighting of the pathophysiological mechanism related features 112 that is performed by the machine learning component 122. For example, certain pathophysiological mechanism related features may be more prognostic for a patient that has high blood pressure than for another patient that has low blood pressure. In another example, a first weighting of first pathophysiological mechanism related features for a first patient with a first heart failure subtype (e.g., HFrEF) is different than a second weighting of the first pathophysiological mechanism related features for a second patient with a second heart failure subtype (e.g., HFpEF), where the first heart failure subtype is different from the second heart failure subtype.

In some embodiments, the medical prognosis of cardiac dysfunction 124 for a patient may be generated by the machine learning component 122 based upon both the plurality of pathophysiological mechanism related features 112 (e.g., the high-impact features 112b) and the clinical information 704 relating to the patient. In such embodiments, the machine learning component 122 may comprise a plurality of different machine learning models 122a-122n that respectively correspond to different combinations of the clinical information 704, so as to account for differences in clinical information between patients. For example, the plurality of different machine learning models 122a-122n may comprise a first machine learning model 122a that has been trained to provide an accurate medical prognosis of heart failure risk for a patient that is a smoker, a second machine learning model 122b that has been trained to provide an accurate medical prognosis of heart failure risk for a patient that has a first heart failure subtype (e.g., HFrEF), a third machine learning model 122c that has been trained to provide an accurate medical prognosis of heart failure risk for a patient that has a second heart failure subtype (e.g., HFpEF), etc. In various embodiments, the medical prognosis of heart failure risk may identify a degree of heart failure the patient has (e.g., low-risk heart failure or high-risk heart failure), a prediction of a time interval until occurrence of a terminal event (e.g., cardiovascular-related mortality), and/or the like.

During operation, clinical information from a patient may be entered into the memory device 102 and then is used to subsequently determine which pathophysiological mechanism related features are provided to the machine learning component 122 and which of the plurality of different machine learning models 122a-122n are used to generate the medical prognosis of cardiac dysfunction 124 for the patient. It has been appreciated that by utilizing the clinical information 704 to identify specific features and/or a specific machine learning model within the machine learning component 122, the disclosed medical assessment apparatus 700 may provide improved results (e.g., a more accurate medical prognosis of cardiac dysfunction for a patient).

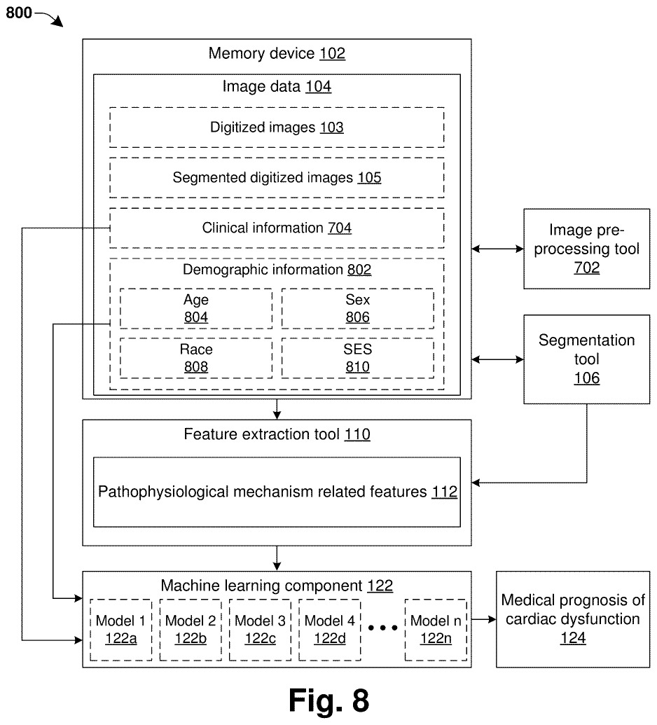

FIG. 8 illustrates some additional embodiments of a medical assessment apparatus 800 comprising a machine learning component configured to utilize a plurality of pathophysiological mechanism related features, clinical information, and/or demographic information to generate a medical prognosis of cardiac dysfunction for a patient.

The medical assessment apparatus 800 comprises a memory device 102 (e.g., electronic memory) configured to store image data 104. The image data 104 comprises one or more digitized images 103 for a patient that may be susceptible to cardiac dysfunction (e.g., heart failure). The one or more digitized images 103 may, for example comprise one or more non-contrast digitized images (e.g., non-contrast MR images), one or more contrast digitized images (e.g., contrast MR images), and/or the like. A segmentation tool 106 is configured to operated upon the digitized images 103 to form one or more segmented digitized images 105. The one or more segmented digitized images 105 may be stored in the memory device 102.

The memory device 102 is further configured to store clinical information 704 and demographic information 802 relating to the patient. In some embodiments, the demographic information 802 may include one or more of an age 804, a sex 806, a race 808, and a socioeconomic status (SES) 810. In some embodiments, the demographic information 802 may further include a geocode of residence, an insurance status, a diagnosis date, and/or the like.

A feature extraction tool 110 is configured to extract a plurality of pathophysiological mechanism related features 112 from the one or more segmented digitized images 105. The plurality of pathophysiological mechanism related features 112 may include features that have been selected to correspond to pathophysiologic mechanisms for heart failure (e.g., heart failure mortality). The plurality of pathophysiological mechanism related features 112 may include one or more of spatial measurements, shape radiomic features, texture radiomic features, and motion-based radiomic features taken from different regions of interest within the one or more segmented digitized images 105.

A machine learning component 122 is configured to utilize the plurality of pathophysiological mechanism related features 112, the clinical information 704, and the demographic information 802 to generate a medical prognosis of cardiac dysfunction 124 for a patient. In some embodiments, the patient may belong to a demographic subgroup (e.g., an African American patient, an Asian American patient, etc.)

In some embodiments, the machine learning component 122 may comprise a plurality of different machine learning models 122a-122n, which have been respectively trained to generate a medical prognosis of cardiac dysfunction for patients that have specific demographic information. For example, the plurality of different machine learning models 122a-122n may comprise a first machine learning model 122a that has been trained to provide an accurate medical prognosis of cardiac dysfunction for a first sex (e.g., that has been trained to be predictive for biological males), a second machine learning model 122b that has been trained to provide an accurate medical prognosis of cardiac dysfunction for a second sex (e.g., that has been trained to be predictive for biological females), a third machine learning model 122c that has been trained to provide an accurate medical prognosis of cardiac dysfunction for a first race (e.g., that has been trained to be predictive for Caucasians), a fourth machine learning model 122d that has been trained to provide an accurate medical prognosis of cardiac dysfunction for a second race (e.g., that has been train to be predictive for African Americans), etc.

In some additional embodiments, the plurality of different machine learning models 122a-122n may also be trained to provide an accurate medical prognosis for different combinations of clinical information and demographic information. In some embodiments, one or more of the plurality of different machine learning models 122a-122n may comprise a model that is generic to clinical information and demographic information. This model may serve as a fallback option for combinations of clinical and/or demographic information that do not have specific models.

During operation, clinical and/or demographic information from a patient may be entered into the memory device 102 and then be used to subsequently determine which pathophysiological mechanism related features 112 are provided to the machine learning component 122 and which of the plurality of different machine learning models 122a-122n are used to generate the medical prognosis of cardiac dysfunction 124 for the patient. It has been appreciated that demographic information 802 (e.g., biological sex, race, age, and SES) plays a role in heart failure pathogenesis and risk prediction. The demographic information 802 stored in the memory device 102 may therefore also affect the pathophysiological mechanism related features 112 used by the machine learning component 122 and/or a weighting of pathophysiological mechanism related features that is performed by the machine learning component 122. Thus, by utilizing the clinical information 704 and the demographic information 802 to identify specific features and/or a specific machine learning model within the machine learning component 122, the disclosed medical assessment apparatus 800 pay provide for improved results.

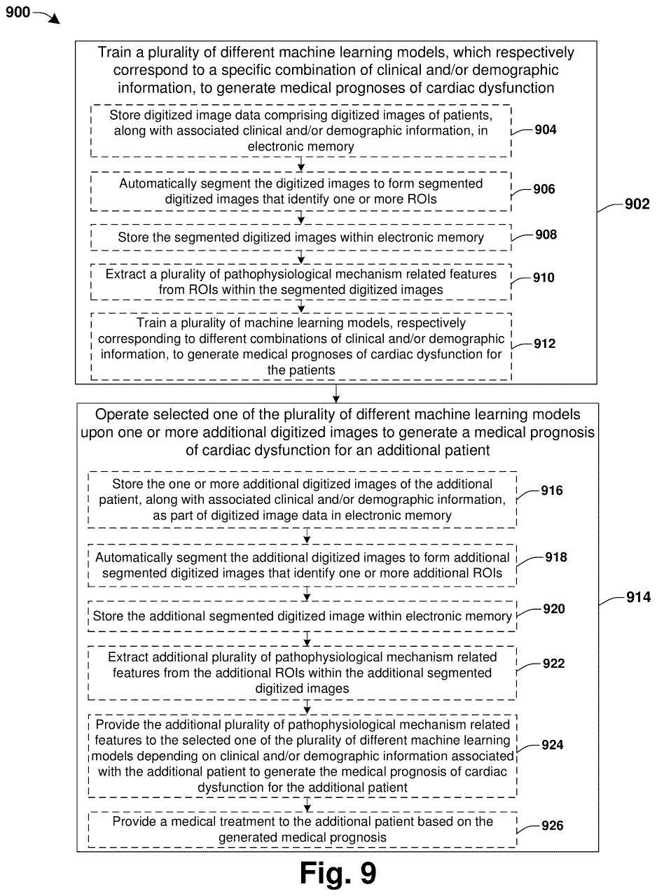

FIG. 9 illustrates a flow diagram showing some additional embodiments of a method 900 of utilizing pathophysiological mechanism related features, clinical information, and/or demographic information to generate a medical prognosis of cardiac dysfunction for a patient.

At act 902, a plurality of different machine learning models, which respectively correspond to a specific combination of clinical and/or demographic information, are trained to generate medical prognoses of cardiac dysfunction. In some embodiments, the act 902 may be performed according to acts 904-912.

At act 904, digitized image data that comprises digitized images of a plurality of patients, and associated clinical and/or demographic information, is stored in electronic memory.

At act 906, the digitized images are automatically segmented to form segmented digitized images that identify one or more regions of interest (ROIs). In some embodiments, the one or more ROIs may comprise one or more of heart tissue, scar tissue, one or more chambers of a heart, such as a right atrium (RA), a right ventricle (RV), a left atrium (LA), a left ventricle (LV) of a heart, and/or the like.

At act 908, the segmented digitized images may be stored within the electronic memory as part of the digitized image data.

At act 910, a plurality of pathophysiological mechanism related features are extracted from the ROIs within the segmented digitized images. In some embodiments, the plurality of pathophysiological mechanism related features comprise one or more of spatial measurements, shape radiomic features, texture radiomic features, and motion-based radiomic features extracted from the ROIs.

At act 912, a plurality of machine learning models, which respectively correspond to different combinations of clinical and/or demographic information, are trained to generate medical prognoses of cardiac dysfunction for the patients. For example, the plurality of machine learning models may comprise models that are trained to provide accurate medical prognoses for patients having specific races, genders, blood pressures, and/or the like.

At act 914, one or more additional digitized images from an additional patient are operated upon by one of the plurality of different machine learning models, depending upon a specific combination of clinical and/or demographic information of the additional patient, to generate a medical prognosis of cardiac dysfunction for the additional patient. In some embodiments, act 914 may be performed according to acts 916-926.

At act 916, the one or more additional digitized images of the addition patient, along with the associated clinical and/or demographic information, are stored in the electronic memory as part of the digitized image data.

At act 918, the one or more additional digitized images are automatically segmented to form additional segmented digitized images that identify one or more additional ROIs. In some embodiments, the one or more additional ROIs may comprise one or more of heart tissue, scar tissue, one or more chambers of a heart, such as a right atrium (RA), a right ventricle (RV), a left atrium (LA), a left ventricle (LV) of a heart, and/or the like.

At act 920, the additional digitized images may be stored within the electronic memory as part of the digitized image data.

At act 922, an additional plurality of pathophysiological mechanism related features are extracted from the one or more additional ROIs within the additional segmented digitized images.

At act 924, the additional plurality of pathophysiological mechanism related features are provided to the selected one of the plurality of different machine learning models depending on the clinical and/or demographic information associated with the additional patient. For example, if the additional patient is male, the additional plurality of pathophysiological related features may be provided to one of the plurality of different machine learning models that has been trained to generate an accurate medical prognosis of cardiac dysfunction for a male patient. If the additional patient is female, the additional plurality of pathophysiological related features may be provided to a different one of the plurality of machine learning models that has been trained to generate an accurate medical prognosis of cardiac dysfunction for a female patient.

At act 926, a medical treatment is provided to the additional patient based on the generated medical prognosis.

It will be appreciated that in some embodiments one of acts 902 and 914 may be excluded from the method 900. For example, in some embodiments, after a trained medical assessment apparatus has been developed, act 902 may be skipped and the method may rather include act 914.

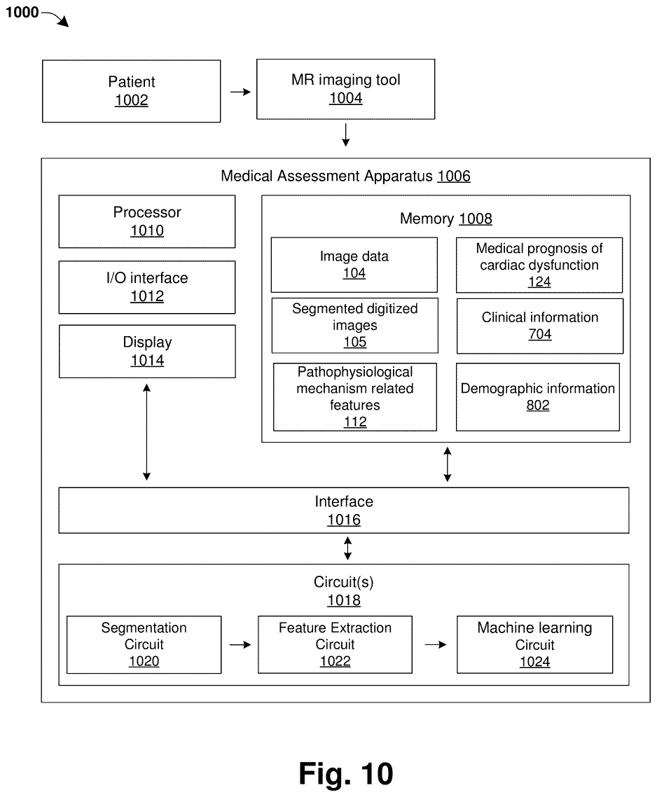

FIG. 10 illustrates a block diagram of some embodiments of a system 1000 comprising a machine learning component configured to utilize a plurality of pathophysiological mechanism related features to generate a medical prognosis of cardiac dysfunction for a patient. In some embodiments, the system 1000 is configured to generate a risk prediction and/or a time to event prediction for a patient that suffers from heart failure-related mortality. In some such embodiments, the patient has been diagnosed with heart failure and the generated risk prediction may, for example, provide a degree of heart failure for the patient (e.g., high-risk heart failure or low-risk heart failure). In further embodiments, the time to event prediction for the patient may, for example, provide a time-to-death prediction (e.g., due to heart failure) for the patient.

The system 1000 comprises a medical assessment apparatus 1006 coupled to an MR imaging tool 1004. The MR imaging tool 1004 is configured to generate one or more images (e.g., MR images) of a patient 1002. In some embodiments, the one or more images may comprise one or more non-contrast images, one or more contrast images, and/or the like. In some embodiments, the MR imaging tool 1004 may comprise a MR scanner.

The medical assessment apparatus 1006 comprises a processor 1010 and a memory 1008. The processor 1010 can, in some embodiments, comprise circuitry such as, but not limited to, one or more single-core or multi-core processors. The processor 1010 can include any combination of general-purposed processors and dedicated processors (e.g., graphics processors, application processors, etc.). The processor(s) 1010 can be coupled with and/or can comprise memory (e.g., memory 1008) or storage and can be configured to execute instructions stored in the memory 1008 or storage to enable various apparatus, applications, or operating systems to perform operations and/or methods discussed herein.

The memory 1008 can be further configured to store image data 104 comprising one or more digitized images (e.g., non-contrast digitized images, contrast digitized images, etc.) obtained by the MR imaging tool 1004. The one or more digitized images may comprise a plurality of pixels or voxels, each pixel or voxel having an associated intensity. In some embodiments, the one or more digitized images may be stored in the memory 1008 as one or more training sets of digitized images for training a classifier and/or one or more validation sets (e.g., test sets) of digitized images.

The medical assessment apparatus 1006 also comprises an input/output (I/O) interface 1012 (e.g., associated with one or more I/O devices), a display 1014, one or more circuits 1018, and an interface 1016 that connects the processor 1010, the memory 1008, the I/O interface 1012, the display 1014, and the one or more circuits 1018. The I/O interface 1012 can be configured to transfer data between the memory 1008, the processor 1010, the one or more circuits 1018, and external devices (e.g., the MR imaging tool 1004).

In some embodiments, the one or more circuits 1018 may comprise hardware components. In other embodiments, the one or more circuits 1018 may comprise software components. The one or more circuits 1018 can comprise a segmentation circuit 1020 (e.g., a deep learning circuit) configured to perform a segmentation operation on one or more segmented digitized images 105 respectively identifying one or more regions of interest (e.g., one or more of a heart, one or more of a muscle tissue, one or more of chamber(s) of a heart, and the like). In some embodiments, the one or more segmented digitized images 105 may comprise binary masks, which may be stored in the memory 1008.

In some additional embodiments, the one or more circuits 1018 may further comprise a feature extraction circuit 1022 configured to extract a plurality of pathophysiological mechanism related features 112 from the one or more segmented digitized images 105. The plurality of pathophysiological mechanism related features 112 may be stored in the memory 1008.

In some embodiments, the one or more circuits 1018 may further comprise a machine learning circuit 1024 configured to operate one or more machine learning models (e.g., a Cox-proportional Hazard model) upon the plurality of pathophysiological mechanism related features 112 to generate a medical prognosis of cardiac dysfunction 124.

In some embodiments, the machine learning circuit 1024 may be configured to selectively operate a plurality of different machine learning models upon different sets of pathophysiological mechanism related features. In such embodiments, the plurality of different machine learning models respectively correspond to a specific combination of clinical information 704 and/or demographic information 802 stored in the memory 1008. During operation, the machine learning circuit 1024 may operate one of the plurality of different machine learning models upon different pathophysiological mechanism related features extracted from one or more images of a patient (e.g., the patient 1002), based on the clinical information and/or demographic information relating to the patient, to generate the medical prognosis of cardiac dysfunction 124 for the patient.

Therefore, the present disclosure relates to a method and associated apparatus that utilizes a plurality of pathophysiological mechanism related features, which have been extract from image data (e.g., magnetic resonance (MR) image), to generate a medical prognosis of cardiac dysfunction.

In some embodiments, the present disclosure relates to a method, the method includes accessing image data stored in a memory, the image data corresponding to a patient; extracting a plurality of pathophysiological mechanism related features from the image data, the plurality of pathophysiological mechanism related features correspond to one or more pathophysiological mechanisms relating to heart failure; and providing the plurality of pathophysiological mechanism related features to a machine learning component, the machine learning component is configured to generate a medical prognosis for the patient using the plurality of pathophysiological mechanism related features.

In other embodiments, the present disclosure relates to an apparatus, the apparatus includes a memory device configured to store image data of a patient, wherein the image data comprises one or more segmented digitized images that identify one or more of muscle tissue, a left ventricle (LV), a left atrium (LA), a right ventricle (RV), and a right atrium (RA); a feature extraction tool configured to extract a plurality of pathophysiological mechanism related features from the one or more segmented digitized images, the plurality of pathophysiological mechanism related features comprise spatial measurements, shape radiomic features, texture radiomic features, and motion-based radiomic features; and a machine learning component configured to generate a risk prediction of heart failure-related mortality for the patient based on the plurality of pathophysiological mechanism related features.

In yet other embodiments, the present disclosure relates to a non-transitory computer-readable medium storing computer executable instructions that, when executed, cause a processor to perform operations, including accessing image data stored in a memory, the image data comprises one or more digitized images corresponding to a patient; extracting a plurality of pathophysiological mechanism related features from the one or more digitized images, the plurality of pathophysiological mechanism related features comprise spatial measurements, shape radiomic features, texture radiomic features, and motion-based radiomic features corresponding to one or more of muscle tissue, a left ventricle (LV) of a heart, a left atrium (LA) of the heart, a right ventricle (RV) of the heart, and a right atrium (RA) of the heart; and providing the plurality of pathophysiological mechanism related features to a machine learning component that has been trained to generate a medical prognosis of heart failure for the patient, the medical prognosis of heart failure includes a time to heart failure-related mortality.

Examples herein can include subject matter such as an apparatus, including a digital whole slide scanner, an MRI system, a CT system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
accessing image data stored in a memory, the image data corresponding to a patient;
extracting a plurality of pathophysiological mechanism related features from the image data, wherein the plurality of pathophysiological mechanism related features correspond to one or more pathophysiological mechanisms relating to heart failure; and
providing the plurality of pathophysiological mechanism related features to a machine learning component, wherein the machine learning component is configured to generate a medical prognosis for the patient using the plurality of pathophysiological mechanism related features.

2. The method of claim 1, wherein the medical prognosis comprises a time to heart failure-related mortality.

3. The method of claim 1, wherein the plurality of pathophysiological mechanism related features comprise spatial measurements, shape radiomic features, texture radiomic features, and motion-based radiomic features.

4. The method of claim 3, wherein the motion-based radiomic features comprise one or more motion-based features of a left ventricle of a heart, a left atrium of the heart, a right ventricle of the heart, a right atrium of the heart, and scar tissue in the heart.

5. The method of claim 1, wherein the plurality of pathophysiological mechanism related features comprise one or more of cardiac remodeling features, cardiac function features, and fibrosis features.

6. The method of claim 5, wherein the cardiac remodeling features comprise one or more of left ventricle (LV) volumes, LV mass, left atrium (LA) volume, right ventricle (RV) size, and right atrium (RA) size, wherein the cardiac function features comprise one or more of LV strain, LA function, RA function, RA strain, and wherein the fibrosis features comprise one or more of scar tissue presence, scar tissue size, and scar tissue characteristics.

7. The method of claim 1, wherein the plurality of pathophysiological mechanism related features are extracted from one or more regions of interest including one or more of muscle tissue, a left ventricle of a heart, a left atrium of the heart, a right ventricle of the heart, and a right atrium of the heart.

8. The method of claim 1, further comprising:
providing the plurality of pathophysiological mechanism related features to a first machine learning model of a plurality of machine learning models within the machine learning component, based upon clinical information relating to the patient, wherein the plurality of machine learning models respectively have been trained to provide accurate medical prognoses of heart failure for a specific combination of clinical information.

9. The method of claim 8, wherein the clinical information comprises a heart failure subtype of the patient.

10. The method of claim 1, wherein the image data comprises one or more cardiovascular magnetic resonance (CMR) image(s).

11. The method of claim 1, further comprising:
preprocessing one or more digitized images in the image data to enhance the one or more digitized images;
segmenting the enhanced one or more digitized images to form one or more segmented digitized images that identify one or more of chambers of a heart and muscle tissue; and
storing the one or more segmented digitized images in the memory as part of the image data.

12. A medical assessment apparatus, comprising:
a memory device configured to store image data of a patient, wherein the image data comprises one or more segmented digitized images that identify one or more of muscle tissue, a left ventricle (LV), a left atrium (LA), a right ventricle (RV), and a right atrium (RA);
a feature extraction tool configured to extract a plurality of pathophysiological mechanism related features from the one or more segmented digitized images, wherein the plurality of pathophysiological mechanism related features comprise spatial measurements, shape radiomic features, texture radiomic features, and motion-based radiomic features; and
a machine learning component configured to generate a risk prediction of heart failure-related mortality for the patient based on the plurality of pathophysiological mechanism related features.

13. The medical assessment apparatus of claim 12, further comprising:
a segmentation tool configured to receive one or more digitized images of the image data from the memory device and generate the one or more segmented digitized images from the one or more digitized images.

14. The medical assessment apparatus of claim 12, wherein the risk prediction of heart failure-related mortality comprises a time to heart failure-related mortality, wherein the image data comprises one or more non-contrast cardiovascular magnetic resonance (CMR) image(s) and one or more contrast CMR image(s).

15. The medical assessment apparatus of claim 12, wherein the memory device is further configured to store clinical information of the patient, the clinical information comprising a heart failure subtype of the patient, wherein the machine learning component comprises a plurality of machine learning models, wherein the plurality of pathophysiological mechanism related features are provided to an individual machine learning model in the plurality of machine learning models depending upon the heart failure subtype of the patient.

16. The medical assessment apparatus of claim 12, wherein the memory device is further configured to store demographic information of the patient, wherein the machine learning component comprises a plurality of machine learning models, the plurality of machine learning models respectively trained to generate the risk prediction of heart failure-related mortality for a specific set of demographic information, wherein the plurality of pathophysiological mechanism related features are provided to an individual machine learning model in the plurality of machine learning models depending upon the demographic information.

17. The medical assessment apparatus of claim 12, wherein the motion-based radiomic features comprise LV strain, LA function, LA strain, RA function, and RA strain.

18. A non-transitory computer-readable medium storing computer executable instructions that, when executed, cause a processor to perform operations, comprising:
accessing image data stored in a memory, wherein the image data comprises one or more digitized images corresponding to a patient;
extracting a plurality of pathophysiological mechanism related features from the one or more digitized images, wherein the plurality of pathophysiological mechanism related features comprise spatial measurements, shape radiomic features, texture radiomic features, and motion-based radiomic features corresponding to one or more of muscle tissue, a left ventricle (LV) of a heart, a left atrium (LA) of the heart, a right ventricle (RV) of the heart, and a right atrium (RA) of the heart; and
providing the plurality of pathophysiological mechanism related features to a machine learning component that has been trained to generate a medical prognosis of heart failure for the patient, wherein the medical prognosis of heart failure includes a time to heart failure-related mortality.

19. The non-transitory computer-readable medium of claim 18, wherein the operations further comprise:
storing clinical information relating to the patient in the memory; and
providing the plurality of pathophysiological mechanism related features to a specific machine learning model within the machine learning component based upon the clinical information relating to the patient.

20. The non-transitory computer-readable medium of claim 18, wherein the operations further comprise:
storing demographic information relating to the patient in the memory; and
providing the plurality of pathophysiological mechanism related features to a specific machine learning model within the machine learning component based upon the demographic information relating to the patient.
